(19) United States
(12) Patent Application Publication
(10) Pub. No.: US 2012/0316518 A1
(43) Pub. Date: Dec. 13, 2012

(54) WOUND DRESSING, CONTAINING NONWOVEN FABRIC AND OINTMENT BASE FOR NEGATIVE PRESSURE THERAPY

(76) Inventors: Pierre Croizat, Herbrechtingen (DE); Axel Eckstein, Heidenheim (DE)

(21) Appl. No.: 13/492,034

(22) Filed: Jun. 8, 2012

Related U.S. Application Data
(60) Provisional application No. 61/494,943, filed on Jun. 9, 2011.

(51) Int. Cl. A61M 35/00; B05D 7/00; B05D 3/12; A61M 1/00

(52) U.S. Cl. 604/304; 604/313; 427/2.31

(57) ABSTRACT

The invention relates to a device for the negative pressure treatment of wounds comprising (a) a covering material for providing a wound space with an air-tight seal; (b) optionally means for connecting a negative pressure source; and (c) a wound dressing containing (c1) a nonwoven material which is wetted with (c2) an ointment base, and also a method for producing a corresponding wound dressing. In addition, the invention relates to the use of an nonwoven material, which is wetted with an ointment base, for application as a wound dressing in the negative pressure treatment of wounds.

The invention relates to a device for the negative pressure treatment of wounds, comprising (a) a covering material for providing a wound space with an air-tight seal; (b) optionally means for connecting a negative pressure source; and (c) a wound dressing containing (c1) a nonwoven material and (c2) an ointment base, and a process for producing a corresponding wound dressing. In addition, the invention relates to the use of a nonwoven material which is wetted with an ointment base, for application as a wound dressing in the negative pressure treatment of wounds.

A wound is understood to mean a separation of the coherence of tissues in the integument of human beings or animals. It may be accompanied by a loss of substance.

Devices for the negative pressure treatment of wounds are known in the state of the art.

WO 1993/009727 A1, for example, describes a device for promoting wound healing by the application of negative pressure to the skin area containing the wound and surrounding the wound. The device according to WO 93/009727 A1 comprises a negative pressure means for generating the negative pressure, an air-tight cover for the wound, which is functionally connected to the negative pressure means, and a wound dressing for positioning on the wound inside the air-tight cover.

Devices for the negative pressure treatment of wounds are commercially available, such as the VivanoTec® ex Paul Hartmann AG. In commercially available devices, a wound dressing is often used which contains an open-celled polymeric foamed material, such as polyvinyl alcohol (PVA) or polyurethane (PU), for example.

The standard commercial foam pads are compressed to different degrees, depending on the negative pressure applied. This can result in a constriction of the passageways needed for removing the exudate from the wound. In addition, it may happen that the foam may adhere to the base of the wound. Newly formed tissue may grow into the foam. This problem is a known complication in the negative pressure treatment of wounds (FDA complaint database). In order to solve this problem, additional wound contact layers are frequently introduced between the foam and base of the wound, such as a film (see, for example, WO2001/85248 A1). However, these additional wound contact layers may reduce the passage of exudate.

Furthermore, when the dressing is changed, stuck and matted foam has to be removed in a time-consuming process, such as by rinsing with Ringer's solution. Tissue that has grown into the foam can lead to tissue traumatisation when the dressing is changed, and may thus disturb the wound healing.

Furthermore, when conventional wound dressings are used, foam particles may penetrate the wound. This problem is aggravated if the wound dressing is trimmed before use, to the size of the wound, because then in particular loose foam particles are created at the edges of the cuts. It has also been found that the standard polymeric foamed materials in some cases stick to the covering film. When the wound dressing sticks to the covering film, this is disadvantageous, especially when the dressing is removed.

The present invention is based on the problem of further improving the treatment of wounds with negative pressure and overcoming the disadvantages of the state of the art. The present invention is intended to provide devices and processes for the treatment of wounds with negative pressure, with which treatment can be performed as effectively and gently as possible.

In particular, the present invention is intended to enable the treatment of wounds with negative pressure, which makes it possible to change a patient's wound dressings at intervals of up to three days, for example. During that time, the patient is intended to have the subjective impression that the wound dressing is comfortable. Pressure irritation and reddening of the skin should be avoided. When the wound dressing is changed after a period of 3 days, for example, as few unpleasant odours as possible should arise. The germ count in the changed wound dressing should be low

The intention is to provide a wound dressing in which sticking to the covering film is avoided as far as possible. The suction force should not decline depending on the distance from the port. The intention is also to provide a wound dressing which allows the user to apply a low negative pressure.

It has unexpectedly been found that the problems could be solved by using a wound dressing containing a nonwoven material and an ointment base. It has surprisingly also been found that a device for negative pressure therapy comprising at least one wound dressing wetted with an ointment base is particularly suitable for the advantageous, i.e. very effective and very gentle, treatment of wounds. By using the wound dressing of the invention it was possible to achieve a better adaptation to the base of the wound. In addition, the pressure irritation felt by the patient from the wound dressing was advantageously reduced.

It has been found that the problems are solved particularly advantageously if a specific nonwoven material and/or a specific ointment base are used and/or if the type of nonwoven material and the type and quantity of the ointment base are matched to one another. As a result, it was possible in particular to achieve a noticeable reduction in the number of particles formed during cutting, and also an advantageous reduction in sticking of the base of the wound to the wound dressing. It has also been found that when the wound dressing of the invention is used, in contrast to the wound dressings of the state of the art, less negative pressure is needed, which is usually felt by the patient to be pleasant.

The subject matter of a first aspect of the invention is therefore a device for the negative pressure treatment of wounds, comprising

(a) a covering material for providing a wound space with an air-tight seal;

(b) optionally means for connecting a negative pressure source; and

(c) a wound dressing, containing

(c1) a nonwoven material, which is wetted with

(c2) an ointment base, which preferably has a dropping point of 20 to 80° C.;

wherein the proportion of ointment base (c2) is 10 to 95% by weight, based on the total weight of the wound dressing.

The subject matter of a second aspect of the invention is a device for the negative pressure treatment of wounds, comprising

(a) a covering material for providing a wound space with an air-tight seal;

(b) optionally means for connecting a negative pressure source; and

(c) a wound dressing, containing

(c1) a nonwoven material, which is wetted with

(c2) an ointment base based on a triglyceride.

The subject matter of a third aspect of the invention is a device for the negative pressure treatment of wounds, comprising

(a) a covering material for providing a wound space with an air-tight seal;

(b) optionally means for connecting a negative pressure source; and

(c) a wound dressing, containing

(c1) a special nonwoven material which has an air permeability of 200 to 5,000 l/(m² sec), measured in accordance with DIN EN ISO 9237, and is wetted with

(c2) an ointment base.

The proportion of ointment base (c2) is preferably 10 to 95% by weight, based on the total weight of the wound dressing.

The invention likewise comprises any combinations of the above-mentioned aspects. Another subject matter of the invention is the use of a nonwoven material which is wetted with an ointment base, preferably with an ointment base with a dropping point of 20 to 80° C., especially with a triglyceride ointment base, as a wound dressing for or in the negative pressure treatment of wounds. This means that a I subject matter of the invention is a nonwoven material which is wetted with a triglyceride ointment base as a wound dressing in the negative pressure treatment of wounds.

Another subject matter of the invention is a method of producing a wound dressing, comprising the steps of

(I) heating an ointment base, preferably to above the dropping point;

(II) introducing the nonwoven material into the heated ointment base, so that the nonwoven material is wetted with the ointment base;

(III) optionally temporarily compressing the nonwoven material, preferably to at least 90% and no more 10% of its original volume, in order to achieve wetting of the surface of the nonwoven material with the ointment base; and

(IV) optionally removing the surplus ointment base, preferably by squeezing out the nonwoven material.

In addition, wound dressings obtainable by the method of the invention are a subject matter of the invention.

A further subject matter of the invention is the use of a nonwoven material (c1) which is wetted with an ointment base (c2) as a wound dressing (c) for or in the negative pressure treatment of wounds, especially burns, wounds caused by mechanical traumatisation, wounds caused by the action of chemicals, wounds caused by disturbances of the metabolism, wounds caused by circulation disorders or wounds caused by decubitus ulcers.

The novel device of the invention and the use of the wound dressing in accordance with the invention are characterised by a number of unexpected advantages.

Wetting the nonwoven material with the ointment base made it possible advantageously to reduce the number of particles which undesirably penetrate the wound.

The use of the wound dressing of the invention improved the atraumatic properties, so that negative pressure treatment, optionally even without any additional wound contact layers, became possible.

Wetting the nonwoven material with the ointment base reduced the subjective feeling of itching in patients and the sense that the nonwoven material was hard during negative pressure treatment. The nonwoven material felt more comfortable, and patient compliance (adherence to the therapy instructions by the patient) was improved.

In addition, it has been shown that despite the use of the ointment base, the suction force does not decline undesirably depending on the port. It has surprisingly been found that with the wound dressing of the invention, it is possible to achieve both a sufficient transport of wound exudate and also an effect supporting wound healing, especially when an ointment base is used which comprises triglycerides and optionally diglycerides. This might be due to the activity of the lipoprotein lipase endogenously present in the wound. The fatty acids released from the triglycerides in the ointment base appear to have a positive effect on wound healing.

An effect supporting wound healing can be achieved with the wound dressing of the invention especially when the wound dressing provides the wound with a sufficient amount of ointment base. Too large an amount of these components may, however, lead to clogging of the pores of the nonwoven material and thus hinder the passage of wound exudate. The amount of ointment base is preferably 10 to 95% by weight, more preferably 30 to 85% by weight, even more preferably 35 to 80% by weight, particularly preferably 40 to 75% by weight, especially 45 to 70% by weight, based on the total weight of the wound dressing.

The manufacturing process of the invention is technically simple and can also be used with (medical) nonwoven materials obtained as a finished product. Wetting is performed, as a result of which the release of particles is reduced in the majority of the areas of nonwoven material when the nonwoven material is cut. In contrast to the process known from the state of the art, in which the impregnation means is added before the foam cures or is synthesised (cf. EP 0 335 669), no undesirable interactions occur between the ointment base, on the one hand, and the nonwoven material on the other hand.

A further advantage of the wound dressing of the invention consists in the fact that the base of the wound can be largely prevented from becoming matted and/or sticking to the wound dressing itself over a period of, for example, up to 3 days. Traumatisation of the wound when changing dressings can be avoided. This enhances the efficacy of the wound treatment. This makes it possible to change a patient's wound dressings at intervals of up to three days, for example. During the period of three days, the wound dressing of the invention was felt by the patient to be comfortable. Pressure irritation, itching and reddening of the skin were avoided in most cases. When the wound dressing was changed after a period of 3 days, there was little unpleasant odour. The germ count in the changed wound dressing was unexpectedly low.

Components (a) to (c) of the device of the invention will be described below.

The device of the invention comprises a covering material (a) for providing a wound space with an air-tight seal. The wound space means the wound and optionally the adjacent region around the wound. An “air-tight seal” here should not be understood as implying that no exchange of gas between the wound space and its surroundings occurs. Rather, “air-tight seal” in this connection means that taking account of the negative pressure pump used, the negative pressure needed for the negative pressure treatment of wounds can be maintained. It is therefore also possible to use covering materials which exhibit a slight gas permeability, provided that the negative pressure needed for the negative pressure treatment of wounds can be maintained.

In a preferred embodiment of the invention, the covering material for providing the wound with an air-tight seal comprises a water-insoluble polymer or a metal film. The covering material is preferably from 10 μm to 10,000 μm, especially from 25 μm to 100 μm, thick.

In a preferred embodiment of the invention, the covering material (a) is a water-insoluble polymer. The water-insoluble polymer preferably has a solubility of 10 mg/l or less, more preferably 1 mg/ml or less, especially from 0.0001 to 1 mg/ml (determined using the column elution method in accordance with EU Directive RL67-548-EEC, Annex V chap. A6). Examples are polyurethane, polyester, polypropylene, polyethylene, polyamide or polyvinyl chloride, polyorganosiloxane (silicone) or a mixture thereof. The polymers mentioned are preferably present here in non-cellular form.

It has been found that a covering material with a specific water vapour permeability is able to solve the problems referred to at the beginning in a particularly advantageous manner. In a preferred embodiment, the covering material therefore has a water vapour permeability of 100 to 2,500 g/m²×24 h, more preferably from 500 to 2,000 g/m²×24 h, even more preferably from 800 to 1,600 g/m²×24 h, especially from 1,050 to 1,450 g/m²×24 h, determined in accordance with DIN EN 13726-2 at 23° C. and 85% relative humidity. Especially the combination of a covering film (a) with the above-mentioned water vapour permeability and a nonwoven material (c1) with the physical properties described below is particularly advantageous.

In a preferred embodiment, the covering material and the means for connecting a negative pressure source are already provided joined together ready for use. It is most particularly preferably that this embodiment should contain a film consisting of one or more water-insoluble polymers which has a self-adhesive edge, because this arrangement considerably facilitates applying the dressing.

The negative pressure therapy device of the invention optionally comprises means (b) for connecting a negative pressure source, i.e. means for producing negative pressure in the wound space. In a preferred embodiment, this is a means (b) for functionally connecting the wound space to a negative pressure source located outside the covering material, so that negative pressure can be created in the wound space and fluids can be withdrawn from the wound space.

The expression “negative pressure in the wound space” refers in connection with the invention to an air pressure within the wound dressing which is lower than the ambient air pressure (atmospheric air pressure). The expression “within the wound dressing” means the space formed between the covering material and the wound.

The pressure difference between the air pressure within the wound dressing and the ambient air pressure is expressed in the context of the invention in mm Hg (millimetres of mercury column). 1 mm Hg corresponds to one Torr or 133.322 Pa (Pascal). In the context of the invention, the negative pressure, i.e. the pressure difference between the air pressure within the wound dressing and the ambient air pressure is expressed as a positive figure in mm Hg.

In one embodiment of the invention, the negative pressure is a negative pressure of at least 20 mm Hg and a maximum of 250 mm Hg, preferably at least 50 mm Hg and a maximum of 150 mm Hg. This negative pressure range has proven advantageous for wound healing. In a preferred embodiment of the invention, the negative pressure is a negative pressure of at least 80 mm Hg and a maximum of 140 mm Hg, more preferably at least 120 mm Hg and a maximum of 130 mm Hg.

As explained above, the device of the invention for the negative pressure treatment of wounds preferably comprises means (b) for connecting a negative pressure source, i.e. means for functionally connecting the wound space to a negative pressure source located outside the covering material.

The functional connection may, for example, be made with a connection line or a negative pressure connector. The person skilled in the art is familiar with negative pressure connectors, which he refers to as “ports”.

In one embodiment, the means (b) is a connection line, preferably a tube, especially a silicone drainage tube. The connection line may be guided through the covering material. Alternatively, the at least one connection line may be guided beneath the edge of the covering material. In both cases, the site where it passes through must be sealed in an air-tight manner so that the desired negative pressure in the dressing can be maintained. Suitable sealants are, for example, an adhesive film, an adhesive mixture, or an adhesive strip. The connection line may, for example, be a hose, a tube or some other body with a cavity.

In a further preferred embodiment, the means (b) is a negative pressure connector (port) which can be attached to one of the inside or outside surfaces of the covering material, with the covering material having suitable apertures for that purpose. In this embodiment too, it must be ensured that there is an air-tight seal either through the passage aperture (port inside) or the area where it is attached (port outside). The seal may, for example, be created with an adhesive film, an adhesive mixture, or an adhesive strip. It is also conceivable that the port itself may possess corresponding attachment means, such as adhesive surfaces. Suitable negative pressure connectors are commercially available. Typically, these are negative pressure connectors which are attached to the outside of the covering material. It is also convenient for the negative pressure connector to have a negative pressure adapter so that it can be connected to the other components of the negative pressure system.

In addition to the above-mentioned components (a) and optionally (b), the device of the invention also comprises (c). The wound dressing (c) which is used in the device of the invention will be described in greater detail below. All the explanations concerning the wound dressing (c), including the nonwoven material (c1) and the ointment bases (c2), relate not only to the device of the invention, but also to the method of the invention for producing the wound dressing and the use of the wound dressing in negative pressure therapy in accordance with the invention.

The wound dressing (c) contains a nonwoven material (c1) and an ointment base (c2).

A nonwoven material usually contains fibres which lie together but are not joined to one another. For this reason the strength of a nonwoven material is preferably due only to the inter-fibre adhesion. The strength referred to can then be influenced by suitable processing methods, so that stabilisation occurs.

In the context of this application, a nonwoven material (c1) is intended to mean a sheet or three-dimensional structure of fibres arranged in alignment or randomly relative to one another, which have been bonded together mechanically and/or thermally and/or chemically.

Nonwoven materials differ fundamentally from woven and knitted materials, which are characterised by the way the individual fibres or yarns are laid, which in turn depends on the method of manufacture.

The nonwoven materials can differ in the type of fibre and their origin, the spinning process to obtain the fibres and the bonding process.

Fibres of natural or synthetic origin or mixtures thereof can be used in the manufacture of nonwoven materials.

The fibres of natural origin include, for example, silk, cellulose, cotton and wool.

The fibres of synthetic origin comprise the synthetic polymers (artificial, or synthetic, fibres). Examples of these are viscose, polyacrylate, polyamide, polyimide, polyamidimide, polyurethane, polyester (especially polyethylene terephthalate and polybutylene terephthalate), polyether esters, polyethers, polyacrylonitrile, polyalkene (especially polyethylene and polypropylene) polyurethane and polytetrafluorethylene. Polyesters are preferable.

The nonwoven materials can be distinguished by the orientation of their fibres. There are nonwoven materials in which the fibres exhibit a preferred direction (fibre-orientated nonwoven materials) and nonwoven materials in which there is no preferred direction of the fibres (random laid nonwovens). In a preferred embodiment, random laid nonwovens are used.

The spinning methods that can be used to obtain the fibres are, for example, the dry spinning method, the wet spinning method, the melt spinning method and the matrix spinning method.

The stabilisation processes which are needed in order to produce the final nonwoven material from the nonwoven starting material may, as mentioned above, be mechanical and/or thermal and/or chemical stabilisation processes.

Mechanically, nonwoven materials can be obtained by stabilisation of the nonwoven starting material/the fibres by means of needling. In this process, needles provided with fine serrations pierce the nonwoven material and tear bundles of fibres with them, which form intertwining loops and result in stabilisation. Different types of needles, e.g. crown needles, may be used for this purpose. Hydroentanglement is often used nowadays instead of the traditional needling process.

Chemical stabilisation of the nonwoven starting material to nonwoven material is usually achieved by adding binders, such as elastomers or plastomers. Chemical stabilisation methods in this context comprise, for example, impregnation with liquid or foam binder(s), imprinting binders or spray stabilisation.

Thermal stabilisation means bonding thermoplastic fibres together or bonding other fibres with the aid of thermoplastic materials. For this purpose, thermoplastic binding fibres can already be mixed in during manufacture of the fibres. Thermal activation can be achieved with hot air or calendars. For some time, it has also been possible to manufacture the nonwoven material by partial welding using ultrasound.

The stabilisation processes can be employed alone or in combination with one another. It is, for example, possible for a nonwoven starting material to be treated with gentle needling on one side and thermal stabilisation on the other in order to obtain a nonwoven material.

In a preferred embodiment, in at least 60% of the fibrous component of the nonwoven material (c1), there are fibres which have a length-to-diameter ratio of at least 350, preferably 400 to 650. It has been found that the ratio specified makes it possible to achieve an advantageous stiffness of the nonwoven material used in the application. With a greater ratio, the nonwoven material is too stiff, whereas it is too yielding with a smaller ratio.

In a further preferred embodiment, the fibres of the nonwoven material have a diameter of 0.1 to 100 μm, preferably 0.25 to 75 μm, more preferably 0.5 to 50 μm, particularly preferably 0.75 to 25 μm, especially 1 to 10 μm.

In a further preferred embodiment, the nonwoven material has a basis weight of 25 to 850 g/m², preferably 50 to 750 g/m², more preferably 100 to 650 g/m², especially 200 to 600 g/m², measured in accordance with DIN EN 29073-1 (specimen area 100 cm² (e.g. 10 cm×10 cm), conditioning to the standardised climate (23° C., 50% r.h.) to constant weight, determination of the weight).

It has been found that nonwoven materials with fibres of the above-mentioned diameter and the above-mentioned basis weight are considered by patients to be very pleasant in terms of wearing comfort.

In a further preferred embodiment, the nonwoven material (c1) has a thickness of 1 to 75 mm, preferably 3 to 50 mm, more preferably 5 to 40 mm, especially 7 to 35 mm.

It has also been found that the problems described above can be solved in an unexpectedly advantageous manner if the nonwoven material (c1) possesses a specific air permeability. In a preferred embodiment, the nonwoven material (c1) has an air permeability of 200 to 5,000 l/(m² sec), more preferably 300 to 3,500 l/(m² sec), even more preferably 350 to 3,000 l/(m² sec), especially 400 to 2,500 l/(m² sec), measured in accordance with DIN EN ISO 9237 (20 mm testing thickness, 20 cm² testing area, 100 Pa differential pressure).

In principle, the nonwoven material (c1) can consist of any of the above-mentioned materials. It should, however, satisfy certain physical requirements, because it has been found that the problems described above can be solved in an unexpectedly advantageous manner if the nonwoven material (c1) possesses a specific tensile strength, especially in the production direction, and a specific stretch, especially against the production direction. In a preferred embodiment, the nonwoven material (c1) has a tensile strength in the production direction of 25 N per 5 cm to 1,000 N per 5 cm, more preferably 100 N per 5 cm to 900 N per 5 cm, even more preferably 200 N per 5 cm to 800 N per 5 cm, measured in accordance with DIN EN 12127. In addition, the nonwoven material (c1) preferably has a stretch, especially against the production direction, of 25% to 750%, more preferably 100% to 700%, even more preferably 200% to 600%, measured in accordance with DIN EN 29073 T3.

It has also been found that the problems described above can be solved in an unexpectedly advantageous manner if the nonwoven material (c1) has a density of between 0.001 and 0.21 g/m³, more preferably between 0.005 and 0.15 g/m³, even more preferably between 0.01 and 0.10 g/m³, especially between 0.012 and 0.05 g/m³, measured in accordance with DIN 53885 (test specimen of sufficient size (preferably 50 mm×50 mm, conditioning to the standardised climate (23° C./50% r.h.) for at least 16 hours, determination of thickness (preferably with Wolf Universal thickness measuring device DM 100) and weight).

It has also been found that the problems described above can be solved in an unexpectedly advantageous manner if the nonwoven material (c1) contains silver in the form of silver ions or in the form of atomic silver. After the production of the nonwoven material (c1), it is preferable for a silver coating to be applied. Alternatively, the silver can already be applied to the nonwoven starting material and hence before stabilisation to the nonwoven material. The nonwoven material (c1) preferably contains 0.000001 to 0.1% by weight, more preferably 0.0001 to 0.01% by weight silver, based on the total weight of the nonwoven material (c1).

In a preferred embodiment, the nonwoven material (c1) is wetted in the dry state with the ointment base (c2). This means that the nonwoven material is preferably not impregnated with an activation solution (such as Ringer's solution), for example.

In principle, the ointment bases known in the art and described in Bauer, Frömming, Führer “Lehrbuch der Pharmazeutischen Technologie”, 8th edition, chapter 12.1 to 12.6, are suitable for use as the ointment base (c2). Ointment bases are accordingly understood to mean spreadable, semi-solid preparations which are in principle suitable for use on the skin or mucous membranes, but which do not (yet) contain any pharmaceutical active agents. The person skilled in the art is familiar with the production of ointment bases; reference may be made to Ph. Eur. 6.0 “Semi-solid preparations for cutaneous application”.

Ointment bases (c2) are preferably used which do not contain an aqueous phase. It is preferable to use hydrophobic, water-absorbing and/or hydrophilic ointment bases. Hydrophobic ointment bases are preferred.

Hydrophobic ointment bases are ones which contain substantially no polar ingredients and therefore substantially do not actively bind water. It is therefore a lipophilic base, in which water can only be incorporated by means of mechanical dispersion. Examples of preferred hydrophobic ointment bases are hydrocarbon bases (e.g. vaseline or vaseline/paraffin mixtures), diglycerides, triglycerides, waxes, polyalkylsiloxane and mixtures thereof. It is particularly preferred for triglycerides or mixtures of triglycerides and diglycerides to be used as the ointment base (c2).

Water-absorbing ointment bases contain lipophilic substances and surfactants. Examples of water-absorbing ointment bases are W/O emulsions (e.g. wool wax) or o/W emulsions.

Hydrophilic ointment bases are preparations which are miscible with water. A preferred example is a polyethylene glycol ointment base (PEG, with a weight-average molecular weight usually of 300 to 4,000 g/mol, preferably 1,500 to 3,000 g/mol).

In addition to the preferred ointment bases described above, the component (c2) also comprises cream bases (hydrophilic and hydrophobic cream bases), gels (hydrophobic gels, hydrophilic gels) and pastes (suspension ointment bases).

The ointment base (c2) ought to be semi-solid in consistency. In order to solve the problems defined at the beginning, it has proven advantageous for the ointment base to have a dropping point of 20 to 80° C., preferably 25 to 55° C., more preferably 30 to 50° C., even more preferably 33 to 48° C. and especially 35 to 45° C. The dropping point is understood in this context to mean the temperature at which the first drop of a melting substance is released from the metal nipple of a dropping point thermometer. In order to determine the dropping point experimentally, reference is made to the following explanations on FIG. 2.

In addition to the dropping point, it has proven advantageous in solving the problems described at the beginning for the ointment base (c2) to satisfy one or more of the following parameters:

acid number from 0.001 to 2.0 mg KOH/g, determined in accordance with Ph. Eur. 6.0, 2.5.1;

iodine value from 0.001 to 3.0 g I2/100 g, determined in accordance with Ph. Eur. 6.0, 2.5.1;

peroxide value from 0.001 to 1.0 mequi O/kg, determined in accordance with Ph. Eur. 6.0, 2.5.5 A;

OH value from 1 to 100, preferably 5 to 90 mg KOH/g, determined in accordance with Ph. Eur. 6.0, 2.5.3;

saponification value from 200 to 350 mg KOH/g, preferably 240 to 300 mg KOH/g, determined in accordance with Ph. Eur. 6.0, 2.5.3;

maximum heavy metal content of 10 ppm, determined in accordance with Ph. Eur. 6.0, 2.4.8.D.

Wherever nothing different is specified in the standards and Ph. Eur. regulations cited in this application, the test methods are generally performed in a standardised climate, i.e. at 23° C. and 50% relative humidity, and at an air pressure of 1,013 mbar.

In a particularly preferred embodiment, triglycerides are used as the ointment base (c2).

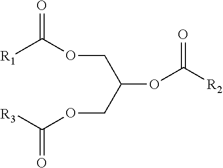

R₁, R₂ and R₃ may be the same or preferably different here.

In a preferred embodiment, the triglyceride contains a glycerine moiety and three C₆-C₂₈ acid moieties, preferably C₈-C₁₈ acid moieties. The acid moieties may be saturated or unsaturated; saturated fatty acids are preferred. The acid moieties may optionally be substituted, e.g. with an hydroxyl group.

Particularly preferably, the acid moieties contain the triglycerides of caprylic acid, capric acid, lauric acid and/or stearic acid. Triglycerides are especially preferred here, wherein the acid moieties fraction contains, and especially consists of, 20 to 40% by weight caprylic acid, 10 to 30% by weight capric acid, 5 to 20% by weight lauric acid and 30 to 50% by weight stearic acid.

In an alternative, particularly preferred embodiment, diglycerides are used as the ointment base (c2).

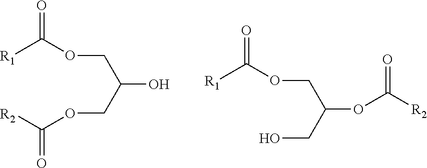

R₁ and R₂ may be the same or preferably different here.

In a preferred embodiment, the diglyceride contains a glycerine moiety and two C₆-C₂₈ acid moieties, preferably C₈-C₁₈ acid moieties. The acid moieties may be saturated or unsaturated; saturated fatty acids are preferred. The acid moieties may optionally be substituted, e.g. with a hydroxyl group. Particularly preferably, the acid moieties of the diglycerides contain isostearic acid, stearic acid, 12-hydroxystearic acid and/or adipic acid.

In a particularly preferred embodiment, the ointment base (c2) contains a mixture of triglycerides and diglycerides. In this context, the ointment base (c2) especially contains 25 to 90% by weight, preferably 45 to 80% by weight triglycerides, and 10 to 75% by weight, preferably 20 to 55% by weight diglycerides.

Similarly, it is preferable that polyethylene glycol (PEG) is also added to the mixture of triglycerides and diglycerides. In particular, the mixture contains triglyceri-des/diglycerides/PEG 1 to 30% by weight, preferably 5 to 20% by weight polyethylene glycol, based on the total weight of the mixture. In this context, PEG with a weight-average molecular weight of 500 to 3,000 g/mol, especially 1,500 to 2,500 g/mol, is preferably used.

A particularly preferred ointment base (c2) contains:

20 to 90% by weight, preferably 55 to 80% by weight triglycerides, especially containing fatty acid residues, selected from caprylic acid, capric acid, lauric acid and/or stearic acid;

5 to 75% by weight, preferably 15 to 45% by weight diglycerides, especially containing fatty acid residues, selected from isostearic acid, stearic acid, 12-hydroxystearic acid and/or adipic acid; and

0 to 30% by weight, preferably 5 to 20% by weight polyethylene glycol with a weight-average molecular weight of 500 to 3,000 g/mol.

In addition to diglycerides and triglycerides, the ointment base (c2) may preferably also contain fatty alcohols, alkoxylated fatty alcohols, fatty acids and alkoxylated fatty acids.

The ointment base (c2) preferably does not contain any monoglycerides.

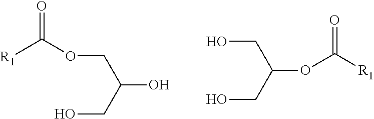

In particular, the ointment base (c2) does not contain any glycerol mono-oleate.

In a preferred embodiment, also substances with an antimicrobial effect can be added to the ointment base (c2). The substances with an antimicrobial effect may, for example, be substances with amino or imino groups. In addition, the substances with an antimicrobial effect may be antimicrobially effective metal cations, especially silver cations, such as a complex of 1-vinyl-2-pyrrolidone with silver cations. Particularly suitable substances with an antimicrobial effect are also biguanide derivatives such as chlorhexidine or polybiguanides, such as polyethylene biguanide (PEB), polytetramethylene biguanide (PTMB) or polyethylene hexamethylene biguanide (PEHMB). A particularly preferred polybiguanide is polyhexamethylene biguanide (PHMB, or polyhexanide). Further suitable substances with an antimicrobial effect are polyguanidines, such as polyhexamethylene guanidine (PHMG), N-octyl-1-[10-(4-octyliminopyridine-1-yl)decyl]pyridine-4-imine (octenidine), quaternary ammonium compounds, such as benzalconium chloride or cetylpyridinium chloride, triazines such as 1-(3-chloroallyl)-3,5,7-triaza-1-azonia-adamantan-chloride or the ammonium compound taurolidine.

Substances with an antimicrobial effect can be added to the ointment base (c2) in an amount of 0 to 15% by weight, preferably 0.1 to 5% by weight, based on the total weight of the ointment base (c2).

In a preferred embodiment, no pharmaceutical active ingredients, especially no substances with antimicrobial effect, are added to the ointment base.

The nonwoven material (c1) is preferably wetted with the ointment base (c2). “Wetting” in this context is understood to mean that the ointment base covers, preferably completely covers, the surfaces of the nonwoven material. In a preferred embodiment, at least 20%, more preferably at least 50%, even more preferably at least 70%, especially at least 90% of the total surface area of the nonwoven material (c1) is covered with ointment base (c2). Wetting of the surface of the nonwoven material (c1) with ointment base (c2) can also be referred to as impregnating.

It has been found that specific amounts of ointment base (c2) are able to solve the problems described at the beginning in a particularly advantageous manner. In a preferred embodiment, the amount of ointment base (c2) is 10 to 95% by weight, more preferably 30 to 85% by weight, even more preferably 35 to 80% by weight, particularly preferably 40 to 75% by weight, especially 45 to 70% by weight, based on the total weight of the wound dressing (c). The total weight of the wound dressing (c) is obtained by adding the weights of components (c1) and (c2).

As a matter of principle, all the explanations concerning preferred embodiments of individual parameters of the nonwoven material (c1) and/or the ointment base (c2) should not be seen in isolation, but also apply in combination with the explanations concerning preferred embodiments of other parameters, or in combination with the explanations concerning the compositions of substances (c1) and (c2).

The wound dressing (c) containing (c1) and (c2) can advantageously be produced by the method of the invention. This comprises the steps of:

(I) heating an ointment base, preferably to above the dropping point;

(II) introducing the nonwoven material into the heated ointment base, so that the nonwoven material is wetted with the ointment base mixture;

(III) optionally temporarily compressing the nonwoven material, preferably to at least 90% and no more 10% of its original volume, in order to achieve wetting of the surface of the nonwoven material with the ointment base; and

(IV) optionally removing the surplus ointment base, preferably by squeezing out the nonwoven material.

In step (I) the ointment base is heated, preferably to about 10 to 30° C. above the dropping point;

In step (II), the nonwoven material is introduced into the heated ointment base and preferably immersed. The dwell time in the mixture from step (II) depends on the size of the nonwoven material and is usually 10 to 100 seconds.

Step (III) may take place during or after, preferably during, immersion.

Temporarily compressing the nonwoven material is understood here to mean that the nonwoven material is compressed manually or by a suitable machine and then released again. In this context, the nonwoven material can be released again immediately after compressing, or may also be held for a short time, such as 1 to 10 seconds, in the compressed state and then released again. After the nonwoven material has been released following the temporary compression, the nonwoven material can then return to its original volume either completely or largely. During the relaxing process, the ointment base is usually absorbed, whereby preferably complete wetting of the surface of the nonwoven material with the ointment base is achieved.

After that, the nonwoven material wetted with the ointment base is removed. Surplus ointment base can be squeezed out in step in step (IV). Squeezing out is preferably performed before the ointment base has cooled to below the dropping point. The squeezing force is preferably selected such that the amount of ointment base is 10 to 95% by weight, more preferably 30 to 85% by weight, even more preferably 35 to 80% by weight, particularly preferably 40 to 75% by weight, especially 45 to 70% by weight, based on the total weight of the wound dressing.

All the explanations provided above on preferred embodiments of components (c), (c1) and (c2) also apply to the method of the invention.

A preferred embodiment is a wound dressing which is obtained by the method of the invention.

In addition, the invention provides a ready-to-use kit for negative-pressure wound treatment comprising the device of the invention, wherein the nonwoven material (c1) is suitable as a wound dressing (c) and is packed ready for use.

One subject matter of the invention is thus a ready-to-use kit for negative pressure wound treatment, comprising

(a) covering material for sealing a wound space in an air-tight manner, i.e. the wound and the wound surroundings;

(b) optionally means for connecting a negative pressure source, preferably means for functionally connecting the wound space to a negative pressure source located outside the covering material, so that negative pressure can be created in the wound space and fluids can be extracted from the wound space, and

(c) a wound dressing packed ready for use, containing

(c1) a nonwoven material, which is wetted with

(c2) an ointment base, which preferably has a dropping point of 20 to 80° C.; The proportion of ointment base (c2) is preferably 10 to 95% by weight, based on the total weight of the wound dressing. It is a preferably a triglyceride ointment base.

The packed wound dressing (c) comprised in the kit is preferably packed so as to be moisture-tight. The ready-to-use wound dressing is preferably provided in sterile form, wherein the sterilisation can be achieved with ethylene oxide gas treatment and/or irradiation. Sterilisation by irradiation is preferred. The irradiation sterilisation can be carried out with beta and/or gamma rays. Beta radiation is preferred. Sterilisation can be achieved by irradiation with a dose of 20 to 60 kGy (20 to 60 kJ/kg). It has been found that a dose of 25 to 40 kGy (25 to 40 kJ/kg) is preferably used. Alternatively, gamma radiation is also preferred.

The kit may include further optional components, such as adhesive means for fixing the dressing, sealing means for producing an air-tight seal in the dressing, pressure sensors, connection elements for pressure sensors, additional tubes, connectors for tubes, disinfectants, skin-care products, pharmaceutical preparations or instructions for use. The kit of the invention preferably also contains scissors, swabs and/or tweezers, especially in sterile form.

The kit may comprise both the at least one wound contact layer, and also at least one additional pressure distribution layer. The kit preferably also comprises a ready-to-use negative pressure unit.

A further subject matter of the invention is the use of the wound dressing (c) described above for or in the negative pressure treatment of wounds, i.e. the use of a nonwoven material (c1), which is wetted with an ointment base (2), for the negative pressure treatment of wounds, especially as a wound dressing (c). A further preferred subject matter of the invention is a wound dressing (c) containing a nonwoven material (c1), an ointment base (c2), for the negative pressure treatment of wounds. A further preferred subject matter of the invention is a method for treating a wound with a wound dressing in accordance with the present application.

All the explanations provided above on preferred embodiments of components (c), (c1) and/or (c2) also apply both individually and in combination to the use in accordance with the invention.

The invention relates, for example, to the use of a nonwoven material (c1), the nonwoven material preferably having a stretch, especially against the production direction, of 25% to 750%, more preferably 100% to 700%, even more preferably 200% to 600%, measured in accordance with DIN EN 29073-T3,

wherein the nonwoven material preferably has a tensile strength, especially in the production direction, of 25 N per 5 cm to 1,000 N per 5 cm, more preferably 100 N per cm to 900 N per 5 cm, even more preferably 200 N per 5 cm to 800 N per 5 cm, measured in accordance with DIN EN 12127;

wherein the nonwoven material preferably has a density of between 0.001 and 0.21 g/cm³, more preferably between 0.005 and 0.15 g/cm³, even more preferably between 0.01 and 0.10 g/cm³, especially between 0.012 and 0.05 g/cm³, measured in accordance with DIN 53885,

wherein the nonwoven material (c1) has an air permeability of 200 to 5,000 l/(m² sec), more preferably 300 to 3500 l/(m² sec), even more preferably 350 to 3,000 l/(m² sec), particularly preferably 400 to 2500 l/(m² sec), especially 500 to 1,000 l/(m² sec), measured in accordance with DIN EN ISO 9237 (20 mm testing thickness, 20 cm² testing area, 100 Pa differential pressure); and

wherein the nonwoven material (c1) has a basis weight of 25 to 850 g/m², preferably 50 to 750 g/m², more preferably 100 to 650 g/m², especially 200 to 600 g/m², measured in accordance with DIN EN 29073-1.

In the use in accordance with the invention, this nonwoven material (c1) is wetted with an ointment base (c2), wherein the ointment base (c2)

has a dropping point of 20 to 80° C., preferably 25 to 55° C., more preferably 30 to 50° C., even more preferably 35 to 48° C. and especially 40 to 45° C.;

is preferably selected from a hydrophobic, water-absorbing and/or hydrophilic ointment base;

preferably contains triglycerides and especially a mixture or triglycerides and diglycerides, e.g. 25 to 90% by weight, preferably 45 to 80% by weight triglycerides, and 10 to 75% by weight, preferably 20 to 55% by weight diglycerides.

Particular advantages achieved by the device of the invention, the kit of the invention or the use or application in accordance with the invention result when the wounds are burns, wounds caused by mechanical traumatisation, chronic wounds caused by the action of chemicals, chronic wounds caused by disturbances of the metabolism, chronic wounds caused by circulation disorders or wounds caused by decubitus ulcers.

In a further preferred embodiment, the wound dressing (c) (containing (c1) and (c2)) can be provided for use in negative pressure therapy in the treatment of a wound caused by a skin transplant. The application comprises the treatment of wounds caused by split skin transplants and full skin transplants, by means of negative pressure therapy. Advantageous effects result from the structure of the nonwoven material (c1) which is wetted with an ointment base (c2), and from the even distribution of pressure. When the wound dressing (c) is used in the treatment of a wound resulting from a skin transplant, the skin graft can be fixed sufficiently firmly in place and at the same time harmful shear forces can be avoided.

The wound dressing (c) described above can advantageously be used in the negative pressure treatment of pressure sores in patients with a body mass index (BMI=body mass divided by body height squared) of less than 18.0, especially with a body mass index of 14 to 17.5. This is especially valid if patients aged more than 60 are concerned. The advantageous effect of the device of the invention or of the kit of the invention is particularly pronounced in these patients.

A further subject matter of the invention is a method for the negative pressure treatment of wounds, comprising the steps of

a) preparing a device in accordance with any of claims 1 to 10;

b) applying the negative pressure dressing to the wound;

c) producing a negative pressure of 20 mm Hg to 250 mm Hg in the wound space for at least 30 minutes and no more than 7 days.

### FIGURES

FIG. 1: Schematic structure of the device the of the invention (side view)

FIG. 2: Device for determining the dropping point

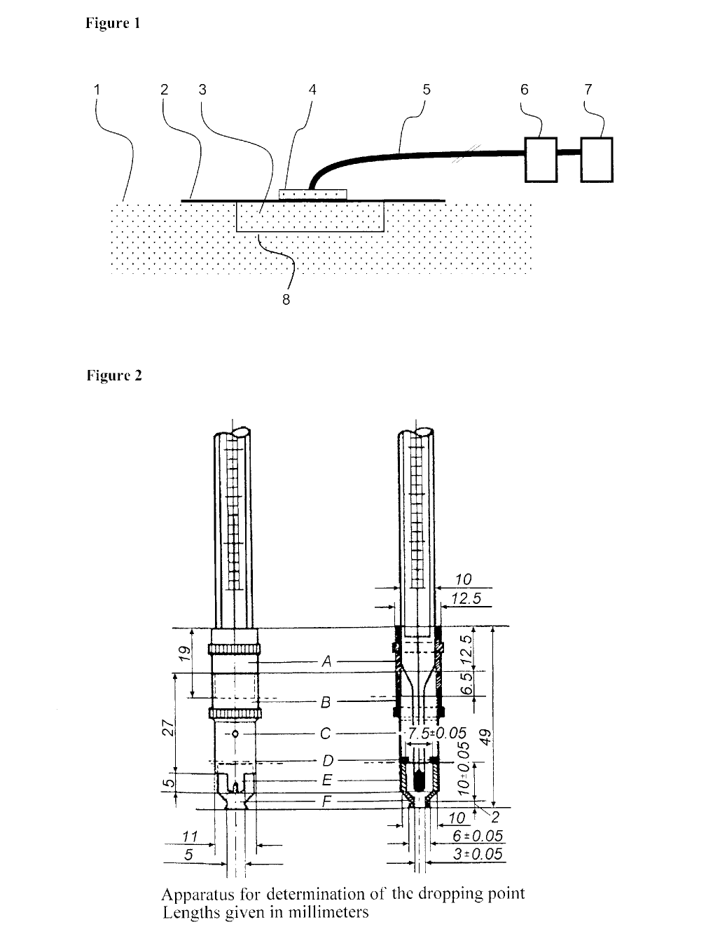

### LEGEND TO FIG. 1

1 wound surroundings (i.e. undamaged skin as a rule)
2 air-impermeable covering material (a)
3 wound dressing (c)=nonwoven material (c1), wetted with ointment base (c2)
4 negative pressure connector (port)
5 negative pressure connection line
6 collection vessel
7 negative pressure unit
8 base of the wound

The device of the invention for the negative pressure treatment of wounds is illustrated in more detail by means of FIG. 1. The invention should not, however, be understood as being reduced to the embodiments illustrated in the drawings or in the description of the drawings. On the contrary, the device of the invention also comprises combinations of the individual features of the alternative embodiments.

FIG. 1 illustrates the schematic structure of the device the of the invention in a side view. The device comprises an air-impermeable covering material (2), means (4-5) for functionally connecting the wound space to a negative pressure source (7) located outside the covering material, and the wound dressing (3), containing nonwoven material (c1) and ointment base (c2). The covering material (2) is attached in the area of the wound surroundings (1), where there is usually undamaged skin The size of the covering material ought to be such that the covering material can be attached outside the wound space in the area of the wound surroundings (1). The covering material (2) can be provided in various dimensions and shapes, such as circular, oval or rectangular. It may also be provided in an irregular shape adapted to the wound. The covering material (2) is usually attached in the area of the wound surroundings (1) and sealed in an air-tight manner. This can be done, for example, by providing the covering material (2) with an adhesive edge. Alternatively, an adhesive substance may either be applied to the edge of the covering material (2) and/or to the intact skin in the area of the wound surroundings. The advantage of this is that it is easier to adapt the covering material to the shape and size of the wound. The negative pressure connector (4) in the preferred embodiment shown here is attached to the outside of the air-impermeable covering material (2), facing away from the wound. In order to functionally connect the wound space to a negative pressure unit (7) located outside the covering material, there must in this arrangement be one or more opening(s) passing through the covering material (2) in the region of the negative pressure connector (4).

In addition, an air-tight seal must be ensured. Such a seal can be created by, for example, applying a film (not shown in FIG. 1) to the top side of the port facing away from the wound and sticking it to the covering material (2). Applying the dressing can be made easier if a port is used in which a suitable fixing and sealing means for fixing the port on the covering material is already present. This is the case, for example, with the commercially available PPM-Drainageport® ex Phametra-Pharma and Medica-Trading GmbH (Herne/Ruhrstadt, Germany).

In a preferred embodiment of the invention, the device for the negative pressure treatment of wounds does not comprise a wound contact layer for insertion between the wound dressing (3) and the wound surface (8).

FIG. 2 shows the device for determining the dropping point. The process for determining the dropping point experimentally is performed as follows:

Test equipment:
Ubbelohde dropping point thermometer
0 to 110° C., calibrated
beaker, 1,000 ml
test tube, approx. 200 mm long, diameter: 40 mm, with pierced bung
magnetic stirrer with heating plate
filter paper blanks 10×10 mm

Reagents:
demineralised water
device for determining the dropping point in accordance with FIG. 2.

The device (see FIG. 2) consists of 2 metal sleeves (A) and (B) screwed together. Sleeve (A) is attached to a mercury thermometer. A metal nipple (F) is loosely attached to the lower part of sleeve (B) by 2 clamping jaws (E). Locking pins (D) 2 mm long fix the position of the nipple accurately. They likewise serve to centre the thermometer. Am opening (C) in the wall of sleeve (B) allows for pressure equalisation.

The dropping surface of the nipple must be flat and the edges of the exit aperture must be at a right angle to it. The lower part of the mercury thermometer is of the shape and dimension show in the illustration. The thermometer permits temperature measurements from 0 to 110° C., its scale division is 1° C. (1 mm each). The mercury bulb of the thermometer has a diameter of 3.5±0.2 mm and a height of 6.0±0.3 mm

The entire device was hung in the middle of a test tube about 200 mm long and with an external diameter of 40 mm by means of a pierced bung, through which the thermometer was inserted. The bung had a notch at the side. The opening of the nipple must be 15 mm above the bottom of the test tube. The entire arrangement was immersed in a 1-litre beaker filled with water. The bottom of the test tube must be about 25 mm above the bottom of the beaker. The water level must reach the upper part of the sleeve (A). A stirrer ensures that the bath retains an even temperature.

The test procedure was conducted as follows.

Unless something else is prescribed, the nipple was filled completely with the unmolten substance to be tested. The excess substance was scraped off both ends of the nipple with a spatula. The sleeves (A) and (B) were screwed together, and the nipple inserted into the sleeve (B) as far as the locking pins. The substance ejected by the thermometer at the nipple opening was scraped off with a spatula. As described above, the device was hung in the water bath. After that, the water bath was heated up in such a way that from about 10° C. below the expected dropping point, the temperature rose by about 1° C. per minute. The temperature was read when the first drop fell off the nipple. The process was repeated at least three times with new samples; a maximum difference of 3° C. between the individual values was permitted.

Evaluation: The dropping point is deemed to be the average of three tests.

The invention will now be illustrated with reference to the following examples.

### EXAMPLES

### Example 1

### Production of the Wound Dressing of the Invention (c)

The ointment base containing a trigylceride/diglyceride mixture (dropping point approx. 40° C.) was heated to 55° C. A nonwoven material (basis weight according to DIN EN 29073-1: 473 g/m²; air permeability according to EN ISO 9237: 556 l/(m² s); thickness: 28 mm) was immersed and squeezed gently. After being removed, the foamed material was squeezed, so that the proportion of ointment base after squeezing was 63% by weight.

### Example 2

### Testing the Wound Dressing of Example 1 in the Wound Simulator

The wound dressing of example 1 was tested in the negative pressure wound simulator (described in DE 10 2008 064 510 A1).

For a “well-conducted” negative pressure therapy simulation, the results of the test had to demonstrate only very small differences in pressure between the wound area (sensor 1) and the port area (sensor 2) and uniform behaviour over a lengthy period, with at the same time a linear pattern with regard to the exudates extracted. Any difference in pressure is due to the wound dressing. The negative pressure was generated using an ATMOS S041 wound drainage suction unit. The exudate was created by a B. Braun Perfusor® F spray pump, which can generate a constant flow. A PPM drainage port system (Herne/Ruhrstadt, Germany) was used.

TABLE 1
Parameters

| | | | Exudate | | |
|---|---|---|---|---|---|
| | Tempera- | | | Viscosity | Wound |
| Wound | ture | Pressure | Flow rate | (mPa · s) | size |
| dressing | (° C.) | (mmHg) | (ml · h⁻¹) | (xanthan) | (cm²) |
| Wound | 23 | 125 | 10 | 98 | 29.6 |
| dressing | | | | | |
| of the | | | | | |
| invention | | | | | |
| in | | | | | |
| accordance | | | | | |
| with | | | | | |
| Example 1 | | | | | |

The xanthan solution was prepared by mixing 1,000 g demineralised water, 2 g xanthan and 0.2 g Allura Red.

The wound dressing was applied to the wound simulator and covered with Hydrofilm® in order to create an air-tight system. Over the artificial wound, a small hole was cut in the film, and a PPM port was attached so that the exudate could flow out of the wound space. This port was connected first to a container in order to collect the exudate, and secondly to the pressure gauge in order to measure the pressure within the port system. The container was connected to a negative pressure pump, and the amount of exudate extracted was measured by determining the weight of the exudate by means of a scale. The other pressure sensor, which was attached inside the wound simulator, measured the pressure inside the simulated wound. The experiment was conducted in accordance with the parameters shown in Table 1.

### Data Analysis

The experiment was over a period of 24 hours. The results are shown below which were obtained for the two above-mentioned pressures (FIG. 3). Sensor 1 measured the pressure within the wound, while sensor 2 determined the pressure within the port system. Averages for the respective pressures and their pressure differences (Table 2).

TABLE 2

| | |
|---|---|
| Sensor 1 average (mmHg): | 121 |
| Sensor 2 average (mmHg): | 123 |
| Average pressure difference | 2 |
| (mmHg): | |

The results show that the pressure difference during the entire experiment remained low, and the behaviour of the pressures remained normal. The exudates calculated and collected displayed a very similar curve, which also demonstrated the success of the experiment.

That leads to the following conclusion.

The amount of exudate collected increased linearly in accordance with expectations, and the pressure difference displayed very good results. This means that in accordance with Example 1, the wound dressing of the invention worked consistently and unexpectedly advantageously in an NPWT simulation process.

1. A device for the negative pressure treatment of wounds comprising,
(a) a covering material for providing a wound space with an air-tight seal;
(b) optionally means for connecting a negative pressure source; and
(c) a wound dressing, containing
(c1) a nonwoven material, which is wetted with
(c2) an ointment base, wherein the proportion of ointment base (c2) is 10 to 95% by weight, based on the total weight of the wound dressing.

2. The device as claimed in claim 1, wherein the ointment base has a dropping point of 20 to 80° C.

3. The device of claim 1, wherein the ointment base (c2) contains a triglyceride, which contains a glycerine moiety and three C₆-C₂₈ acid moieties, preferably C₈-C₁₈ acid moieties.

4. The device of claim 1, wherein the ointment base (c2) contains a diglyceride, which contains a glycerine moiety and two C₄-C₂₈ acid moieties, preferably C₆-C₁₈ acid moieties.

5. The device of claim 1, wherein the nonwoven material (c1) has a thickness of 1 to 75 mm.

6. The device of claim 1, wherein the individual fibres of the nonwoven material (c1) have a thickness of 0.25 to 75 mm.

7. The device of claim 1, wherein the nonwoven material (c1) has a basis weight of 25 to 850 g/m², measured in accordance with DIN EN 29073-1.

8. The device of claim 1, wherein the nonwoven material (c1) has an air permeability of 200 to 5,0001/(m² sec), measured in accordance with DIN EN ISO 9237.

9. The device of claim 1, wherein the nonwoven material (c1) has a density of between 0.001 and 0.21 g/cm³ aufweist, measured in accordance with DIN 53885.

10. The device of claim 1, wherein the covering material (c1) has a water vapour permeability of 100 to 2,500 g/m²×24 h, measured in accordance with DIN EN 13726-2.

11. A method for producing a wound dressing, comprising the steps of
(I) heating an ointment base, preferably to above the dropping point;
(II) introducing the nonwoven material into the heated ointment base, so that the nonwoven material is wetted with the ointment base;
(III) optionally temporarily compressing the nonwoven material, preferably to at least 90% and no more 10% of its original volume, in order to achieve wetting of the surface of the nonwoven material with the ointment base; and
(IV) optionally removing the surplus ointment base, preferably by squeezing out the nonwoven material.

12. The device as claimed in claim 11, wherein the squeezing force is selected such that amount of ointment base is 10 to 95% by weight, based on the total weight of the wound dressing.

13. A wound dressing obtainable by a process as claimed in claim 11.

14. A nonwoven material which is wetted with a triglyceride ointment base for use as a wound dressing in the negative pressure treatment of wounds.

15. The wound dressing as claimed in claim 14, wherein the wounds are burns, wounds caused by mechanical traumatisation, wounds caused by the action of chemicals, wounds caused by disturbances of the metabolism, wounds caused by circulation disorders or wounds caused by decubitus ulcers.
